(19) United States
(12) Patent Application Publication
(10) Pub. No.: US 2019/0167338 A1
(43) Pub. Date: Jun. 6, 2019

(54) TREATMENT TOOL AND TREATMENT SYSTEM

(71) Applicant: OLYMPUS CORPORATION, Tokyo (JP)

(72) Inventor: Shinji YASUNAGA, Tokyo (JP)

(73) Assignee: OLYMPUS CORPORATION, Tokyo (JP)

(21) Appl. No.: 16/267,557

(22) Filed: Feb. 5, 2019

Related U.S. Application Data
(63) Continuation of application No. PCT/JP2016/078310, filed on Sep. 26, 2016.

(51) Int. Cl. A61B 18/08; A61B 18/14

(52) U.S. Cl. CPC A61B 18/085; A61B 2018/00791; A61B 18/1445

(57) ABSTRACT

A treatment tool includes: a first jaw including a first holding surface; a second jaw including a second holding surface; a first wiring pattern that includes a first heat-generating portion; a first heating plate; a second wiring pattern that includes a second heat-generating portion; and a second heating plate. The first heat-generating portion is provided at a first area when the first holding surface is divided into two areas where the first area and a second area are arranged, and the second heat-generating portion is provided at a second projection area when the second holding surface is divided into two areas where a first projection area onto which the first area is projected and the second projection area onto which the second area is projected in a closed state where the first holding surface and the second holding surface are faced each other.

### CROSS REFERENCES TO RELATED APPLICATIONS

This application is a continuation of PCT international application Ser. No. PCT/JP2016/078310 filed on Sep. 26, 2016 which designates the United States, incorporated herein by reference.

### BACKGROUND

### 1. Technical Field

The present disclosure relates to a treatment tool and a treatment system.

### 2. Related Art

In the related art, there are known treatment tools for treating (joining (or inosculating), separating, or the like) living tissue by applying energy to the living tissue (for example, see Japanese Laid-open Patent Publication No. 2014-124491).

The treatment tool (energy treatment tool) according to Japanese Laid-open Patent Publication No. 2014-124491 includes first and second jaws (first and second holding members) that hold living tissue. Furthermore, each of the first and second jaws is provided with an energy applying structure that generates thermal energy and applies the thermal energy to the living tissue.

The energy applying structure includes a wiring pattern (SUS pattern) and a heating plate (first and second high-frequency electrode) described below.

The wiring pattern includes an electric resistance pattern that generates heat with an applied current and a lead connecting portion that is electrically connected to the electric resistance pattern. Furthermore, the lead connecting portion is connected to a lead, and a current is applied to the electric resistance pattern via the lead and the lead connecting portion so that the electric resistance pattern generates heat.

The heating plate is formed of a conductor such as copper. Furthermore, the heating plate transmits the heat from the electric resistance pattern to the living tissue (applies thermal energy to the living tissue).

### SUMMARY

In some embodiments, a treatment tool includes: a first jaw including a first holding surface; a second jaw including a second holding surface to hold living tissue with the first holding surface; a first wiring pattern that is provided on the first holding surface and that includes a first heat-generating portion where a resistance value per unit length in a longitudinal direction connecting a distal end and a proximal end of the first jaw is higher than resistance values of other areas, the first heat-generating portion being configured to generate heat with an applied current; a first heating plate that is disposed to face the first holding surface, the first heating plate being configured to transmit heat from the first wiring pattern to the living tissue by bringing into contact with the living tissue; a second wiring pattern that is provided on the second holding surface and that includes a second heat-generating portion where a resistance value per unit length in a longitudinal direction connecting a distal end and a proximal end of the second jaw is higher than resistance values of other areas, the second heat-generating portion being configured to generate heat with an applied current; and a second heating plate that is disposed to face the second holding surface, the second heating plate being configured to transmit heat from the second wiring pattern to the living tissue by bringing into contact with the living tissue. The first heat-generating portion is provided at a first area when the first holding surface is divided into two areas where the first area and a second area are arranged parallel in the longitudinal direction connecting the distal end and the proximal end of the first jaw, and the second heat-generating portion is provided at a second projection area when the second holding surface is divided into two areas where a first projection area onto which the first area is projected and the second projection area onto which the second area is projected in a closed state where the first holding surface and the second holding surface are faced each other.

In some embodiments, a treatment system includes: the above-mentioned treatment tool; and an applied-current controller configured to apply a current to each of the first wiring pattern and the second wiring pattern, calculate a temperature based on a resistance value of each of the first wiring pattern and the second wiring pattern when applying the current, and execute an applied-current control such that the temperature becomes a target temperature.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram that schematically illustrates a treatment system according to a first embodiment of the disclosure;

FIG. 2 is a diagram that illustrates the distal end part of the treatment tool;

FIG. 3 is a diagram that illustrates the distal end part of the treatment tool;

FIG. 4 is a diagram that illustrates a first energy-applying structure;

FIG. 5 is a diagram that illustrates the first energy-applying structure;

FIG. 6 is a diagram that illustrates a second energy-applying structure;

FIG. 7 is a diagram that illustrates operation for opening and closing first and second jaws;

FIG. 8 is a diagram that illustrates operation for opening and closing the first and second jaws;

FIG. 9A is a diagram that illustrates the positional relationship between the first and second wiring patterns in the closed state where the first and second holding surfaces are faced each other;

FIG. 9B is a diagram that illustrates the positional relationship between the first and second wiring patterns in the closed state where the first and second holding surfaces are faced each other;

FIG. 10 is a block diagram that illustrates the configuration of a control device;

FIG. 11 is a flowchart that illustrates operation of the control device;

FIG. 12A is a diagram that illustrates a first heater according to a second embodiment of the disclosure;

FIG. 12B is a diagram that illustrates a second heater according to the second embodiment of the disclosure;

FIG. 13A is a diagram that illustrates a first heater according to a third embodiment of the disclosure;

FIG. 13B is a diagram that illustrates a second heater according to the third embodiment of the disclosure;

FIG. 14A is a diagram that illustrates a first heater according to a fourth embodiment of the disclosure; and

FIG. 14B is a diagram that illustrates a second heater according to the fourth embodiment of the disclosure.

### DETAILED DESCRIPTION

With reference to the drawings, an aspect (hereinafter, embodiment) for implementing the disclosure is explained below. Furthermore, the disclosure is not limited to the embodiments described below. Moreover, the identical components are attached with the same reference numeral in description of the drawings.

### First Embodiment

### Schematic Configuration of a Treatment System

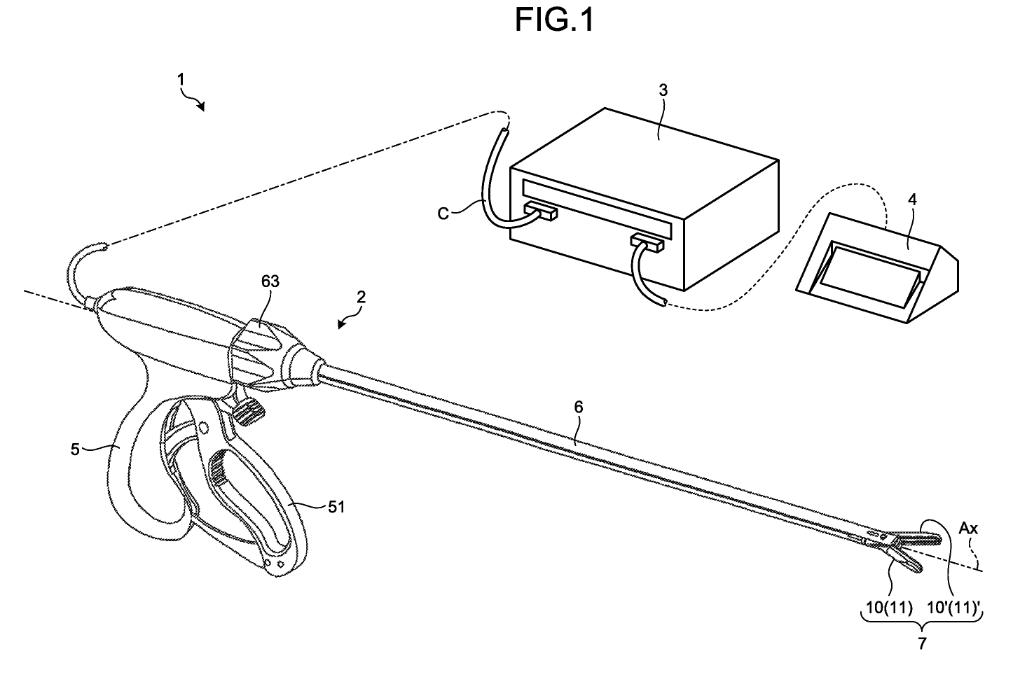

FIG. 1 is a diagram that schematically illustrates a treatment system 1 according to a first embodiment of the disclosure.

The treatment system 1 applies thermal energy to living tissue, which is the target for treatment, so as to give treatment (joining (or inosculation), separation, or the like) to the living tissue. As illustrated in FIG. 1, the treatment system 1 includes a treatment tool 2, a control device 3, and a foot switch 4.

Configuration of the Treatment Tool

The treatment tool 2 is a linear-type surgical treatment tool for giving treatment to living tissue through for example an abdominal wall. As illustrated in FIG. 1, the treatment tool 2 includes an operating handle 5, a shaft 6, and a holding portion 7.

The operating handle 5 is a portion that is held with the operator's hand. Furthermore, as illustrated in FIG. 1, the operating handle 5 is provided with an operating knob 51 that opens and closes first and second jaws 11, 11′ included in the holding portion 7.

Furthermore, in FIG. 1, the structure and the shape indicated by the reference numeral without “′” are substantially the same as the structure and the shape indicated by the reference numeral with “′” added thereto. The same holds for the subsequent drawings.

Configuration of the Shaft

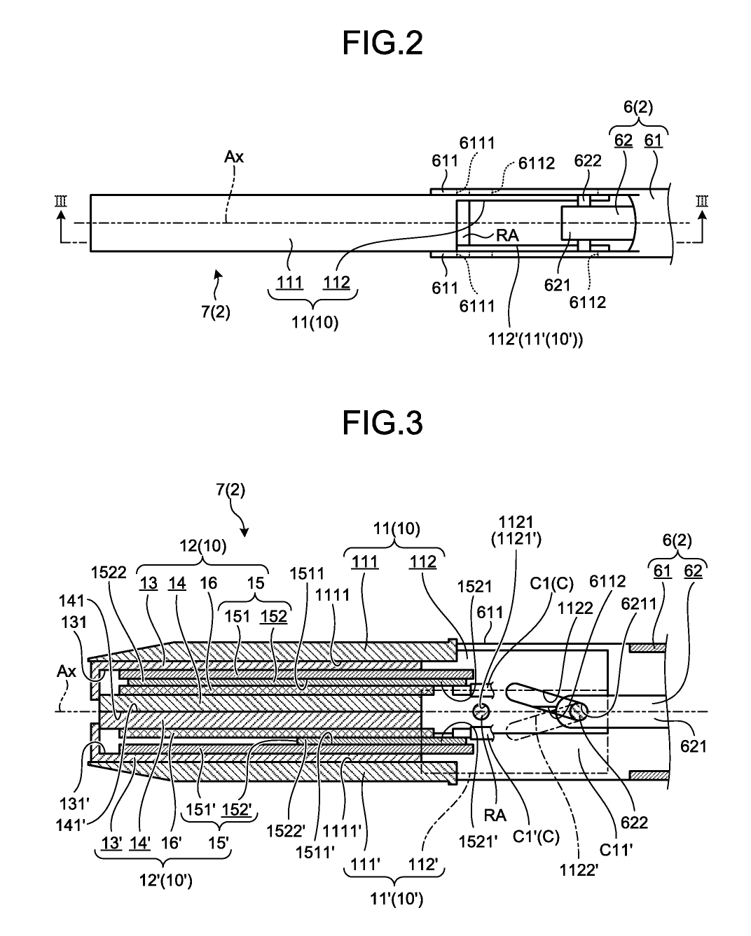

FIG. 2 and FIG. 3 are diagrams that illustrate the distal end part of the treatment tool 2. Specifically, FIG. 2 is a diagram when the distal end part of the treatment tool 2 is viewed from the side of the first jaw 11. For the convenience of explanation, illustration of first and second leads C1, C1′ is omitted from FIG. 2. FIG. 3 is a cross-sectional view taken along the line III-III of FIG. 2.

As illustrated in FIG. 1, the shaft 6 is an elongated member along a central axis Ax; one end thereof is coupled to the operating handle 5 through a rotary support member 63, and the other end thereof pivotally supports the first and the second jaws 11, 11′ such that they may be opened and closed. As illustrated in FIG. 2 or FIG. 3, the shaft 6 includes a cylindrical portion 61 and a rod 62.

Here, the rotary support member 63 supports the shaft 6, and it is attached such that it is rotatable relative to the operating handle 5 with the central axis Ax as a center. Specifically, the rotary support member 63 is rotated in accordance with operation of the operator so that the shaft 6 and the first and the second jaws 11, 11′ attached to the shaft 6 are rotated together with the rotary support member 63 with the central axis Ax as a center.

The cylindrical portion 61 has substantially a cylindrical shape, one end thereof being coupled to the rotary support member 63, and the other end thereof supporting the first and the second jaws 11, 11′ such that they may be opened and closed.

Inside the cylindrical portion 61, an electric cable C (FIG. 1) coupled to the control device 3 is disposed from one end side to the other end side through the operating handle 5 and the rotary support member 63. Furthermore, FIG. 3 illustrates part of the first leads C1 in pair and the second leads C1′ in pair included in the electric cable C.

Furthermore, the other end of the cylindrical portion 61 is provided with a pair of pivotally support portions 611 each protruding toward the distal end (the left side in FIG. 2 and FIG. 3) of the treatment tool 2.

Each of the pivotally support portions 611 in pair is elongated and is shaped like substantially a flat plate. Furthermore, the pivotally support portions 611 in pair extend in a longitudinal direction along the central axis Ax and are faced each other in a vertical direction of FIG. 2.

The pivotally support portions 611 in pair have the same shape. Therefore, the shape of one of the pivotally support portions 611 is explained.

As illustrated in FIG. 2, the pivotally support portion 611 is provided with a first shaft-bearing hole 6111 on the distal end side (the left side in FIG. 2) relative to the center position in a longitudinal direction of the pivotally support portion 611, penetrating through the two sides of the pivotally support portion 611 and having a rotary shaft RA inserted therethrough.

Furthermore, as illustrated in FIG. 2 or FIG. 3, the pivotally support portion 611 is provided with a first track hole 6112 on the proximal end side (the right side in FIG. 2 and FIG. 3) relative to the first shaft-bearing hole 6111, penetrating through the two sides of the pivotally support portion 611 and extending along the central axis Ax.

The rod 62 is disposed inside the cylindrical portion 61 and is moved back and forth along the central axis Ax in accordance with operator's operation on the operating knob 51. That is, the rod 62 forms part of an opening/closing system that opens and closes the first and the second jaws 11, 11′. As illustrated in FIG. 2 or FIG. 3, the rod 62 includes a rod main body 621 and a shaft portion 622.

The rod main body 621 is a portion that is composed of an elongated rod-like member and is moved back and forth along the central axis Ax in accordance with operator's operation on the operating knob 51. Furthermore, the distal end side (the left side in FIG. 2 and FIG. 3) of the rod main body 621 is provided with a through-hole 6211, penetrating in a direction perpendicular to the central axis Ax and having the shaft portion 622 inserted therethrough.

The shaft portion 622 has a cylindrical shape, and it is inserted through the through-hole 6211 of the rod main body 621. Furthermore, as illustrated in FIG. 2, while the shaft portion 622 is inserted through the through-hole 6211, both ends of the shaft portion 622 protrude outward from the rod main body 621. Moreover, both ends of the shaft portion 622 protruding outward from the rod main body 621 are inserted through the first track holes 6112 of the pivotally support portions 611 in pair and second track holes 1122, 1122′ (FIG. 3) of the first and the second jaws 11, 11′.

Configuration of the Holding Portion

The holding portion 7 is a portion that holds the living tissue and gives treatment to the living tissue. As illustrated in FIG. 2 or FIG. 3, the holding portion 7 includes: a first holding portion 10 including the first jaw 11 and a first energy-applying structure 12; and a second holding portion 10′ including the second jaw 11′ and a second energy-applying structure 12′.

Configuration of the First Jaw

The first jaw 11 is a portion that is supported pivotally and rotatably by the pivotally support portions 611 in pair through the rotary shaft RA. As illustrated in FIG. 2 or FIG. 3, the first jaw 11 includes a jaw main body 111 and a jaw connecting portion 112.

As illustrated in FIG. 2, the jaw main body 111 is elongated and is shaped like substantially a flat plate with a width dimension (a length dimension in a lateral direction) slightly smaller than the separate dimension between the pivotally support portions 611 in pair. Furthermore, one surface of the jaw main body 111 functions as a first holding surface 1111 (FIG. 3) to which the first energy-applying structure 12 is attached.

The jaw connecting portion 112 is a portion that couples the first jaw 11 to the cylindrical portion 61. The jaw connecting portion 112 has an elongated and substantially flat-plate like shape with its longitudinal direction along the longitudinal direction of the jaw main body 111, and it is integrally formed with the upper side of the jaw main body 111 in FIG. 2 at one end side (the right side in FIG. 2 and FIG. 3) while it is perpendicular to the jaw main body 111.

As illustrated in FIG. 3, the jaw connecting portion 112 is provided with a second shaft-bearing hole 1121 on the distal end side (the left side in FIG. 3) relative to the center position in the longitudinal direction of the jaw connecting portion 112, penetrating through the two sides of the jaw connecting portion 112. Specifically, the jaw connecting portion 112 abuts the inner surface of the pivotally support portion 611, which is one of the pivotally support portions 611 in pair, and the rotary shaft RA is inserted through each of the first shaft-bearing hole 6111 and the second shaft-bearing hole 1121, whereby the first jaw 11 is pivotally supported such that it is rotatable relative to the cylindrical portion 61 (the pivotally support portions 611 in pair) with the rotary shaft RA as a center.

Furthermore, as illustrated in FIG. 3, the jaw connecting portion 112 is provided with the second track hole 1122 on the proximal end side (the right side in FIG. 3) relative to the second shaft-bearing hole 1121, penetrating through the two sides of the jaw connecting portion 112 and extending in a direction crossing the central axis Ax.

Specifically, the second track hole 1122 is shaped such that it is tilted upward in FIG. 3 as it is closer to the second shaft-bearing hole 1121. Furthermore, in the state (the closed state where the first and the second holding surfaces 1111, 1111′ are faced each other) illustrated in FIG. 3, the right end of the second track hole 1122 in FIG. 3 is set such that it is in the same level as the first track hole 6112. Specifically, in the state illustrated in FIG. 3, the level of the second track hole 1122 gradually becomes higher relative to the first track hole 6112 as it is closer to the second shaft-bearing hole 1121. Moreover, the end of the shaft portion 622 is inserted through the second track hole 1122.

Configuration of the First Energy-Applying Structure

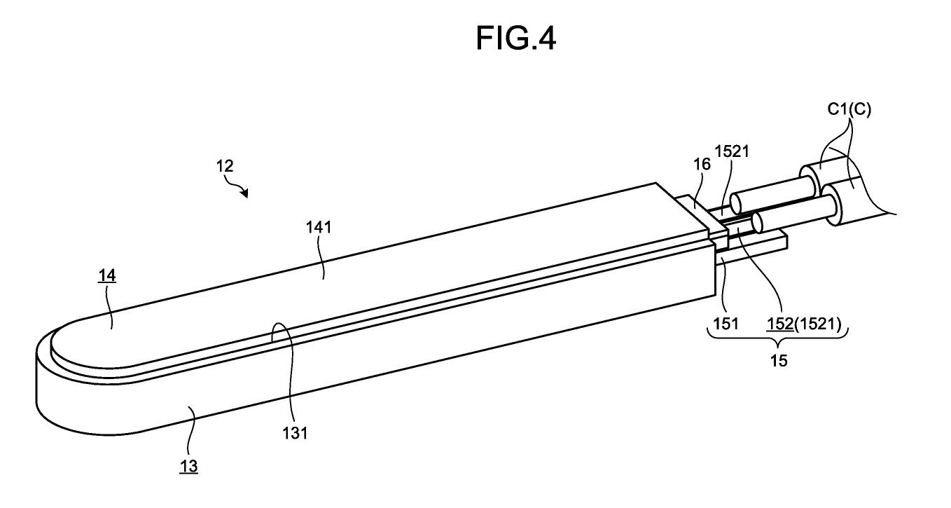

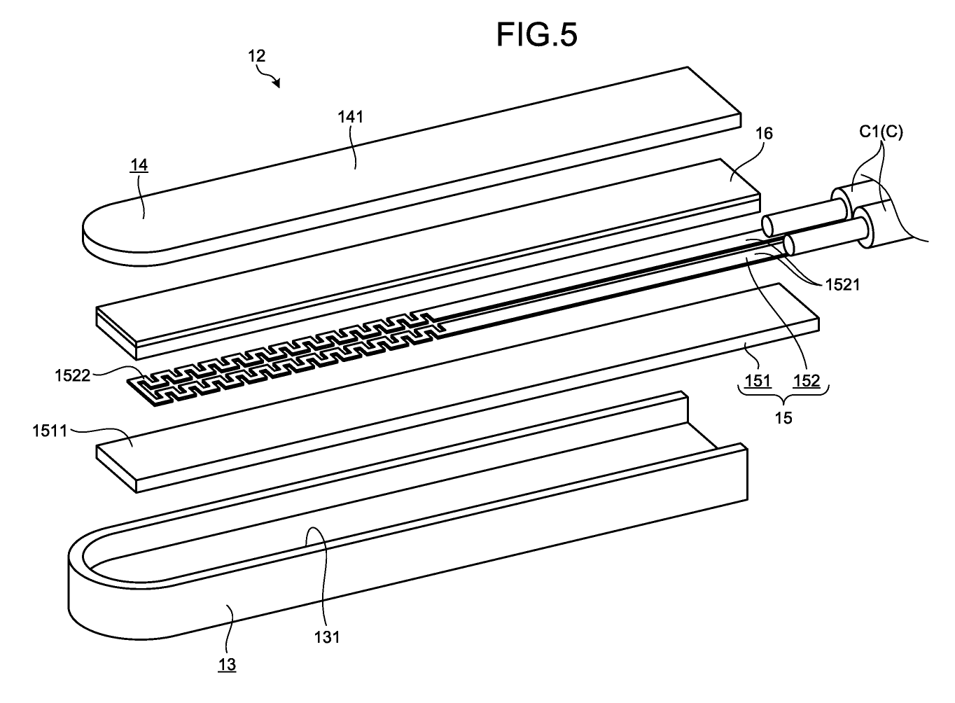

FIG. 4 and FIG. 5 are diagrams that illustrate the first energy-applying structure 12. Specifically, FIG. 4 is a perspective view when the first energy-applying structure 12 is seen from a first treatment surface 141 that is brought into contact with the living tissue. FIG. 5 is an exploded perspective view of FIG. 4.

As illustrated in FIG. 4 or FIG. 5, the first energy-applying structure 12 includes a first cover member 13, a first heating plate 14, a first heater 15, a first adhesive sheet 16, and the first leads C1 in pair.

The first cover member 13 has substantially a cuboidal shape extending along the central axis Ax of the cylindrical portion 61 (extending in the longitudinal direction (the horizontal direction in FIG. 2, FIG. 3) connecting the distal end and the proximal end of the first jaw 11 (the first holding surface 1111)). Furthermore, at substantially the center position of the first cover member 13 in a width direction, a first recessed portion 131 is provided, extending from one end (the extreme right in FIG. 4, FIG. 5) of the first cover member 13 to the other end side in the longitudinal direction of the first cover member 13.

Moreover, as illustrated in FIG. 4, the first heating plate 14, the first heater 15, and the first adhesive sheet 16 are provided in the first recessed portion 131.

The above-described first cover member 13 is molded with a resin material such as fluorine resin.

The first heating plate 14 is an elongated thin plate composed of a material such as copper and extending in the longitudinal direction (the horizontal direction in FIG. 4, FIG. 5) of the first cover member 13. Furthermore, while the holding portion 7 holds the living tissue, the first treatment surface 141 (the upper surface in FIG. 4, FIG. 5), which is the front surface of the first heating plate 14, is brought into contact with the living tissue so as to transmit heat from the first heater 15 to the living tissue (apply thermal energy to the living tissue).

Here, the planar shape of the first heating plate 14 is specified such that it is substantially the same as the planar shape of the first recessed portion 131.

The first heater 15 functions as a sheet heater that partially generates heat and applies the generated heat to the first heating plate 14. As illustrated in FIG. 4 or FIG. 5, the first heater 15 includes a first board 151 and a first wiring pattern 152.

The first board 151 is an elongated sheet composed of polyimide, which is a material with an insulating property and extending in the longitudinal direction of the first cover member 13.

Furthermore, as the material for the first board 151, not only polyimide but also material with high heat resistance and insulating properties, such as aluminum nitride, alumina, glass, or zirconia, may be used.

Here, the width dimension of the first board 151 is specified such that it is slightly smaller than the width dimension of the first heating plate 14. Furthermore, the length dimension (the length dimension in the longitudinal direction) of the first board 151 is specified such that it is longer than the length dimension (the length dimension in the longitudinal direction) of the first heating plate 14.

Moreover, the first board 151 may be composed of an electric conductive material. In such a case, insulating coating may be applied to be electrically insulated from the first wiring pattern 152.

The first wiring pattern 152 is processed of stainless steel (SUS304) that is an electric conductive material, and as illustrated in FIG. 4 or FIG. 5, it includes a pair of first connecting portions 1521 and a first electric resistance pattern 1522 (FIG. 5). Furthermore, the first wiring pattern 152 is bonded to a first surface 1511 (FIG. 5) of the first board 151 due to thermal compression.

Furthermore, as the material for the first wiring pattern 152, not only stainless steel (SUS304) but also other stainless steel materials (e.g., 400 series) or electric conductive material such as platinum or tungsten may be used. Moreover, not only the configuration that the first wiring pattern 152 is bonded to the first surface 1511 of the first board 151 due to thermal compression but also the configuration that it is formed on the first surface 1511 due to vapor deposition, or the like, may be used.

As illustrated in FIG. 4 or FIG. 5, the first connecting portions 1521 in pair are provided such that they extend in the longitudinal direction of the first board 151 with a constant line width and they are faced each other in a width direction of the first board 151. Furthermore, the first connecting portions 1521 in pair are connected (bonded) to the first leads C1 in pair, respectively.

One end of the first electric resistance pattern 1522 is connected (electrically connected) to one of the first connecting portions 1521, serpentines from the end in a wavelike fashion with a constant line width, extends in a U shape that follows the outer edge shape of the first board 151, and the other end is connected (electrically connected) to the other one of the first connecting portions 1521.

According to the first embodiment, the line width of the first electric resistance pattern 1522 is set smaller than the line width of the first connecting portions 1521 in pair. Moreover, the thickness dimension of each of the first connecting portions 1521 in pair and the first electric resistance pattern 1522 is set to be identical. That is, the resistance value of the first electric resistance pattern 1522 per unit length in the longitudinal direction of the first board 151 is set larger than the resistance value of the pair of the first connecting portions 1521.

Furthermore, the first electric resistance pattern 1522 generates heat when the control device 3 applies a voltage (applies a current) to the pair of the first connecting portions 1521 through the pair of the first leads C1.

Here, the first electric resistance pattern 1522 corresponds to a first heat-generating portion according to the disclosure.

As illustrated in FIG. 4 or FIG. 5, the first adhesive sheet 16 is disposed between the first heating plate 14 and the first heater 15, and it adhesively attaches the back surface (the surface on the opposite side of the first treatment surface 141) of the first heating plate 14 and the first surface 1511 of the first board 151 in a state where part of the first heater 15 protrudes from the first heating plate 14. The first adhesive sheet 16 is an elongated (elongated shape extending in the longitudinal direction of the first cover member 13) sheet having desirable heat conductivity and insulating property, resistance to a high temperature, and adherence property, and it is formed by mixing a filler (non-electric conductive material) having high thermal conductivity, such as alumina, boron nitride, graphite, or aluminum nitride, with epoxy or polyurethane resin.

Here, the width dimension of the first adhesive sheet 16 is set to be substantially the same as the width dimension of the first board 151. Furthermore, the length dimension (the length dimension in the longitudinal direction) of the first adhesive sheet 16 is set longer than the length dimension (the length dimension in the longitudinal direction) of the first heating plate 14 and shorter than the length dimension (the length dimension in the longitudinal direction) of the first board 151.

Furthermore, the first heating plate 14 is provided so as to cover the entire area of the first electric resistance pattern 1522. Moreover, the first adhesive sheet 16 is provided so as to cover the entire area of the first electric resistance pattern 1522 and cover part of the pair of the first connecting portions 1521. Specifically, the first adhesive sheet 16 is provided such that it protrudes to the right side in FIG. 4 and FIG. 5 relative to the first heating plate 14. Furthermore, the pair of the first leads C1 is electrically connected to an area of the pair of the first connecting portions 1521 that are not covered with the first adhesive sheet 16.

Configuration of the Second Jaw

The second jaw 11′ has the same configuration and shape as those of the first jaw 11, is faced the first jaw 11, and is pivotally and rotatably supported by the pair of the pivotally support portions 611 through the rotary shaft RA in the posture of the reversed first jaw 11.

As the second jaw 11′ has the same structure and shape as those of the first jaw 11, the same structure as that of the first jaw 11 is attached with the reference numeral having “′” added thereto and its explanation is omitted.

Configuration of the Second Energy-Applying Structure

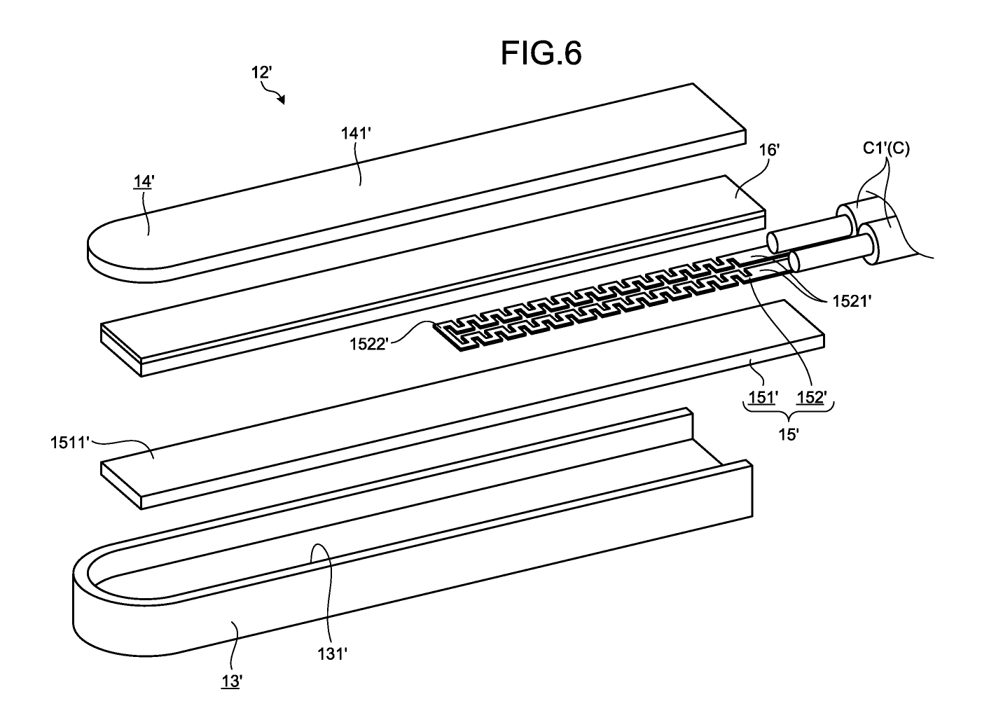

FIG. 6 is a diagram that illustrates the second energy-applying structure 12′. Specifically, FIG. 6 is an exploded perspective view of the second energy-applying structure 12′ when seen from the side of a second treatment surface 141′.

The second energy-applying structure 12′ has substantially the same configuration and shape as those of the first energy-applying structure 12, is faced the first energy-applying structure 12, and is attached to the second holding surface 1111′ of the second jaw 11′ in the posture of the reversed first energy-applying structure 12.

Specifically, as illustrated in FIG. 6, the second energy-applying structure 12′ includes a second cover member 13′ (including a second recessed portion 131′), a second heating plate 14′ (including the second treatment surface 141′), a second heater 15′ (a second board 151′ (including a first surface 1511′) and a second wiring pattern 152′ (including a pair of second connecting portions 1521′ and a second electric resistance pattern 1522′)), a second adhesive sheet 16′, and the pair of the second leads C1′, which correspond, in the first energy-applying structure 12, the first cover member 13 (including the first recessed portion 131), the first heating plate 14 (including the first treatment surface 141), the first heater 15 (the first board 151 (including the first surface 1511) and the first wiring pattern 152 (including the pair of the first connecting portions 1521 and the first electric resistance pattern 1522)), the first adhesive sheet 16, and the pair of the first leads C1.

Here, the second electric resistance pattern 1522′ corresponds to a second heat-generating portion according to the disclosure.

Here, as illustrated in FIG. 6, the second wiring pattern 152′ is specified such that the length dimension (the length dimension in the longitudinal direction of the second board 151′ (the first board 151)) is different from that of the first wiring pattern 152 (FIG. 5). Specifically, in a closed state where the first and the second holding surfaces 1111, 1111′ are faced each other, the first and the second wiring patterns 152, 152′ are asymmetric with respect to a virtual plane that is positioned between the first and the second holding surfaces 1111, 1111′ and parallel to the first and the second holding surfaces 1111, 1111′.

Furthermore, an explanation is given later of the positional relationship between the first and the second wiring patterns 152, 152′ in the closed state where the first and the second holding surfaces 1111, 1111′ are faced each other.

Operation for Opening and Closing the First and Second Jaws

Next, operation for opening and closing the above-described first and second jaws 11, 11′ is explained.

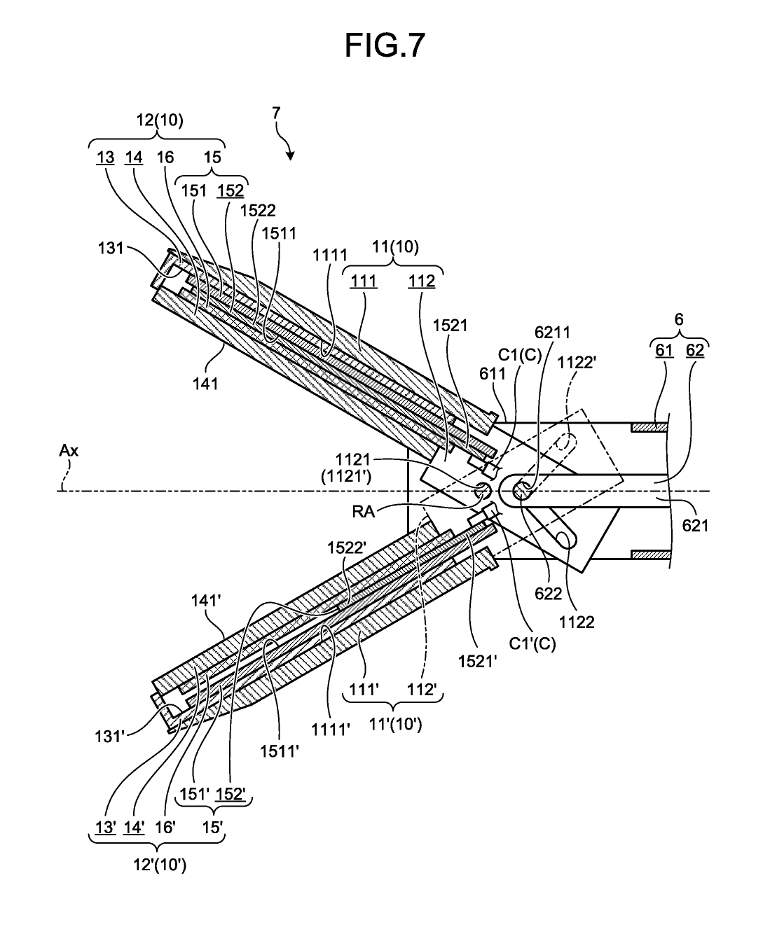

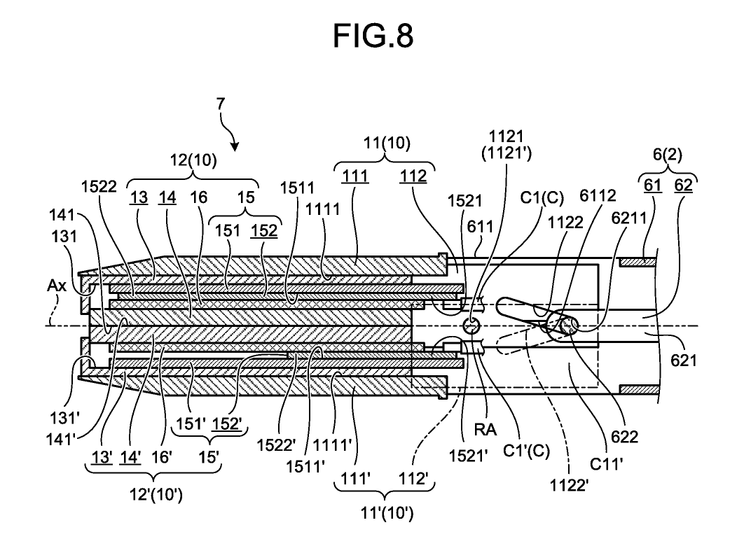

FIG. 7 and FIG. 8 are diagrams that illustrate operation for opening and closing the first and the second jaws 11, 11′. Specifically, FIG. 7 is a cross-sectional view that corresponds to FIG. 3, and it illustrates “the opened state” where the first and the second jaws 11, 11′ are opened. FIG. 8 is the state that corresponds to FIG. 3, and it illustrates a state where the first and the second jaws 11, 11′ are closed, i.e., “the closed state” where the first and the second holding surfaces 1111, 1111′ are faced each other.

In the “opened state” illustrated in FIG. 7, when the operator operates the operating knob 51, the rod 62 moves to the side (the right side in FIG. 7, FIG. 8) of the operating handle 5. Due to the movement of the rod 62, the shaft portion 622 moves from the left side to the right side in FIG. 7 or FIG. 8 within each of the first track holes 6112 and each of the second track holes 1122, 1122′.

Here, each of the first track holes 6112 provided in the cylindrical portion 61 is set so as to extend along the central axis Ax, as described above. Conversely, as described above, the second track hole 1122 provided in the first jaw 11 is set such that its level gradually becomes high relative to each of the first track holes 6112 as it moves to the left side in FIG. 7 or FIG. 8. Furthermore, the second jaw 11′ has the posture of the reversed first jaw 11. Therefore, the level of the second track hole 1122′ provided in the second jaw 11′ gradually becomes lower relative to each of the first track holes 6112 as it moves to the left side in FIG. 7 or FIG. 8.

Therefore, when the shaft portion 622 moves from the left side to the right side in FIG. 6 or FIG. 7 within each of the first track holes 6112 and each of the second track holes 1122, 1122′, it presses the edge portions of the second track holes 1122, 1122′ during move. Then, the first and the second jaws 11, 11′ rotate around the rotary shaft RA in a direction in which the first and the second energy-applying structures 12, 12′ move close to each other and finally enters the “closed state” illustrated in FIG. 8.

In the “closed state” illustrated in FIG. 8, when the operator cancels operation of the operating knob 51, the rod 62 moves from the right side to the left side in FIG. 7 or FIG. 8, contrary to the above. Then, in accordance with the movement of the rod 62, the first and the second jaws 11, 11′ rotate around the rotary shaft RA in a direction in which the first and the second energy-applying structures 12, 12′ separate from each other, contrary to the above, and finally enters the “opened state” illustrated in FIG. 7.

Positional Relationship Between the First and Second Wiring Patterns

Next, the positional relationship between the first and the second wiring patterns 152, 152′ in the “closed state” illustrated in FIG. 8 is explained.

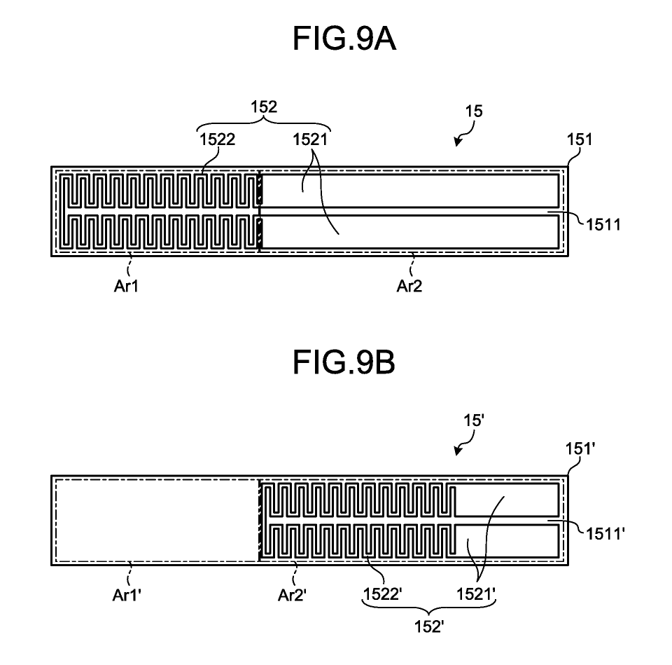

FIG. 9A and FIG. 9B are diagrams that illustrate the positional relationship between the first and the second wiring patterns 152, 152′ in the closed state where the first and the second holding surfaces 1111, 1111′ are faced each other. Specifically, FIG. 9A is a diagram of the first heater 15 when viewed from the side of the first wiring pattern 152. FIG. 9B is a diagram of the second heater 15′ when viewed from the side of the second wiring pattern 152′.

Here, on the first surface 1511 of the first board 151, two areas arranged parallel in the longitudinal direction of the first board 151 are a first area Ar1 and a second area Ar2 (FIG. 9A). The first area Ar1 is positioned at the distal end side (the left side in FIG. 9A) of the first jaw 11 relative to the second area Ar2.

Furthermore, as illustrated in FIG. 9A, the first wiring pattern 152 is provided such that the pair of the first connecting portions 1521 is positioned at the second area Ar2 and the first electric resistance pattern 1522 is positioned at the first area Ar1.

Furthermore, in the “closed state” illustrated in FIG. 8, a first projection area Ar1′ is the area of the first surface 1511′ of the second board 151′ onto which the first area Ar1 is projected, and a second projection area Ar2′ is the area of the first surface 1511′ onto which the second area Ar2 is projected (FIG. 9B).

Furthermore, as illustrated in FIG. 9B, the second wiring pattern 152′ is provided such that it is positioned at the second projection area Ar2′. That is, the second wiring pattern 152′ is not present at the first projection area Ar1′.

Therefore, in the “closed state” illustrated in FIG. 8, the first electric resistance pattern 1522 is not faced the second wiring pattern 152′ (the second electric resistance pattern 1522′). Furthermore, the second electric resistance pattern 1522′ is opposed to the pair of the first connecting portions 1521.

Configuration of the Control Device and the Foot Switch

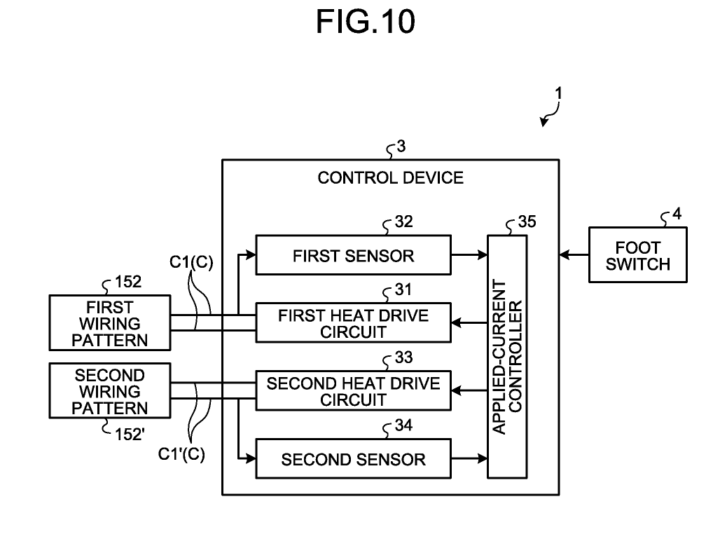

FIG. 10 is a block diagram that illustrates the configuration of the control device 3.

Here, FIG. 10 principally illustrates the relevant part of the disclosure as the configuration of the control device 3.

The foot switch 4 receives a first user operation to shift the treatment tool 2 from a standby state (a standby state for giving treatment to the living tissue by stopping the supply of electric power output to the first and the second wiring patterns 152, 152′) to a treatment state (a state for giving treatment to the living tissue by starting the supply of electric power output to the first and the second wiring patterns 152, 152′) by being pressed (ON) by the operator's foot. Also, the foot switch 4 receives a second user operation to shift the treatment tool 2 from the treatment state to the standby state by the operator's foot separated (OFF) from the foot switch 4. Then, the foot switch 4 outputs signals corresponding the first and second user operations to the control device 3.

Moreover, the configuration to receive the first and second user operations is not limited to the foot switch 4, and a manually operated switch, or the like, may be used.

The control device 3 controls overall operations of the treatment tool 2. As illustrated in FIG. 10, the control device 3 includes a first heat drive circuit 31, a first sensor 32, a second heat drive circuit 33, a second sensor 34, and an applied-current controller 35.

The first heat drive circuit 31 applies a voltage (applies a current) to the first wiring pattern 152 through the pair of the first leads C1 under the control of the applied-current controller 35.

The first sensor 32 detects the current value and the voltage value supplied (electrically applied) to the first wiring pattern 152 from the first heat drive circuit 31. Then, the first sensor 32 outputs the signal corresponding to the detected current value and voltage value to the applied-current controller 35.

The second heat drive circuit 33 applies a voltage (applies a current) to the second wiring pattern 152′ through the pair of the second leads C1′ under the control of the applied-current controller 35.

The second sensor 34 detects the current value and the voltage value supplied (electrically applied) to the second wiring pattern 152′ from the second heat drive circuit 33. Then, the second sensor 34 outputs signals corresponding to the detected current value and voltage value to the applied-current controller 35.

The applied-current controller 35 includes a CPU (Central Processing Unit), or the like, and it controls operation of the treatment tool 2 in accordance with predetermined control programs.

More specifically, the applied-current controller 35 switches the treatment tool 2 to the treatment state when the foot switch 4 is turned on (when the foot switch 4 receives the first user operation). Then, the applied-current controller 35 determines the temperature (hereafter, referred to as first heater temperature) of the first electric resistance pattern 1522 and the temperature (hereafter, referred to as second heater temperature) of the second electric resistance pattern 1522′ and supplies the necessary output value (electric power value) to each of the first and the second wiring patterns 152, 152′ through the pair of the first leads C1 and the pair of the second leads C1′ so that the first and the second electric resistance patterns 1522, 1522′ have the target temperature (executes feedback control).

Here, the first and second heater temperatures used for the feedback control are temperatures calculated as described below.

Specifically, the resistance value of the first wiring pattern 152 is acquired based on the current value and the voltage value detected by the first sensor 32 (the current value and the voltage value supplied (electrically applied) to the first wiring pattern 152 from the first heat drive circuit 31). Then, the resistance value of the first wiring pattern 152 is converted into a temperature by using the relationship between the resistance value and the temperature of the first wiring pattern 152, previously calculated from experiments, and sets the temperature as the first heater temperature.

Furthermore, the resistance value of the second wiring pattern 152′ is acquired based on the current value and the voltage value detected by the second sensor 34 (the current value and the voltage value supplied (electrically applied) to the second wiring pattern 152′ from the second heat drive circuit 33). Then, the resistance value of the second wiring pattern 152′ is converted into a temperature by using the relationship between the resistance value and the temperature of the second wiring pattern 152′, previously calculated from experiments, and sets the temperature as the second heater temperature.

Furthermore, when the foot switch 4 is turned off (when the foot switch 4 receives the second user operation), the applied-current controller 35 switches the treatment tool 2 to the standby state.

Operation of the Control Device

Next, operation of the above-described control device 3 is explained.

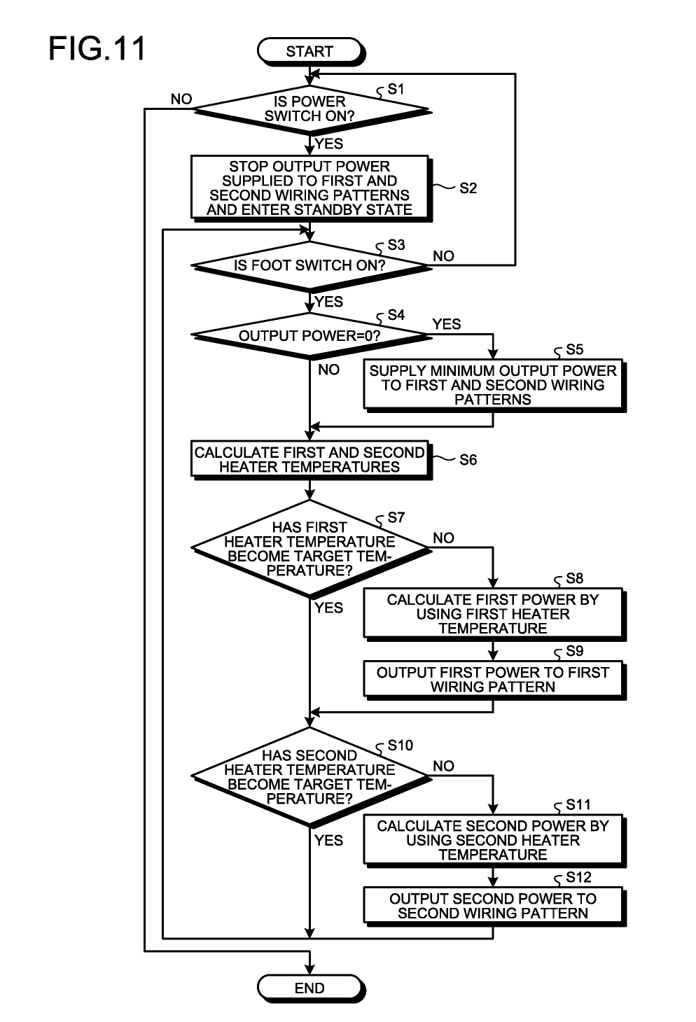

FIG. 11 is a flowchart that illustrates operation of the control device 3.

After the operator turns on the power switch (not illustrated) of the control device 3 (Step S1: Yes), the applied-current controller 35 switches the treatment tool 2 to the standby state (Step S2).

Specifically, at Step S2, the applied-current controller 35 stops the supply of electric power output to the first and the second wiring patterns 152, 152′ via the first and the second heat drive circuits 31, 33.

Then, the operator holds the treatment tool 2 with hand and inserts the distal end part (the holding portion 7 and part of the shaft 6) of the treatment tool 2 into the abdominal cavity through the abdominal wall by using a trocar, or the like. Also, the operator operates the operating knob 51 to hold the living tissue, which is the target for treatment, with the holding portion 7.

After Step S2, the applied-current controller 35 determines whether the foot switch 4 has been turned on due to the first user operation of the operator (Step S3).

When it is determined that the foot switch 4 has been turned off (or the off state continues) due to the second user operation of the operator (Step S3: No), the control device 3 returns to Step S1.

Conversely, when it is determined that the foot switch 4 has been turned on (or the on state continues) (Step S3: Yes), the applied-current controller 35 switches the treatment tool 2 to the treatment state (Step S4 to S12).

First, the applied-current controller 35 determines whether the output power supplied to the first and the second wiring patterns 152, 152′ is zero (Step S4).

When it is determined that the output power supplied to the first and the second wiring patterns 152, 152′ is zero (Step S4: Yes), the applied-current controller 35 supplies the minimum output power (e.g., 0.1 W) to the first and the second wiring patterns 152, 152′ via the first and the second heat drive circuits 31, 33 so as to calculate the first and second heater temperatures (detect the current value and the voltage value with the first and the second sensors 32, 34) (Step S5).

When it is determined that the output power supplied to the first and the second wiring patterns 152, 152′ is not zero (Step S4: No) or after Step S5, the applied-current controller 35 calculates the first and second heater temperatures based on the current value and the voltage value detected by the first and the second sensors 32, 34 (Step S6).

After Step S6, the applied-current controller 35 determines whether the first heater temperature has become the target temperature (the first heater temperature has reached the target temperature) (Step S7).

When it is determined that the first heater temperature has not become the target temperature (Step S7: No), the applied-current controller 35 calculates first power by using the first heater temperature (Step S8). Here, for calculation of the first power, typical PID (Proportional-Integral-Differential) control, or the like, is used. Then, after Step S8, the applied-current controller 35 outputs (supplies) the first power to the first wiring pattern 152 via the first heat drive circuit 31 (Step S9).

When it is determined that the first heater temperature has become the target temperature (Step S7: Yes) or after Step S9, the applied-current controller 35 determines whether the second heater temperature has become the target temperature (the second heater temperature has reached the target temperature) (Step S10).

When it is determined that the second heater temperature has not become the target temperature (Step S10: No), the applied-current controller 35 calculates second power by using the second heater temperature (Step S11). Furthermore, for calculation of the second power, typical PID control, or the like, is used as is the case with calculation of the first power. Then, after Step S11, the applied-current controller 35 outputs (supplies) the second power to the second wiring pattern 152′ via the second heat drive circuit 33 (Step S12).

When it is determined that the second heater temperature has become the target temperature (Step S10: Yes) or after Step S12, the control device 3 returns to Step S3.

Due to the above-described feedback control, each of the first and the second heating plates 14, 14′ is heated, and the heat of the first and the second heating plates 14, 14′ gives treatment to the living tissue held by the first and the second heating plates 14, 14′.

In the above-described treatment tool 2 according to the first embodiment, the first electric resistance pattern 1522 is provided at the first area Ar1 on the distal end side. Conversely, the second electric resistance pattern 1522′ is provided at the second projection area Ar2′ on the proximal end side.

Therefore, when the living tissue is held by part of the first and the second jaws 11, 11′ on the distal end side (hereafter, described as being held at the distal end side), substantially the entire first electric resistance pattern 1522 is covered with the living tissue. Furthermore, when the living tissue is held by part of the first and the second jaws 11, 11′ on the proximal end side (hereafter, described as being held at the proximal end side), substantially the entire second electric resistance pattern 1522′ is covered with the living tissue.

Therefore, when the above-described feedback control is executed so that the first and the second electric resistance patterns 1522, 1522′ have the target temperature, the living tissue may be heated at the target temperature in any case when it is held at the distal end side or when it is held at the proximal end side.

As described above, with the treatment tool 2 according to the first embodiment, there are advantages such that the living tissue may be heated at the desired temperature and the treatment time may be shortened.

### Second Embodiment

Next, a second embodiment of the disclosure is explained.

In the following explanation, the same structure as that in the above-described first embodiment is attached with the same reference numeral, and its detailed explanations are omitted or simplified.

A treatment tool according to the second embodiment is different from the treatment tool 2 explained according to the above-described first embodiment in the configurations of the first and the second heaters 15, 15′. Therefore, only the configurations of the first and second heaters according to the second embodiment are explained below.

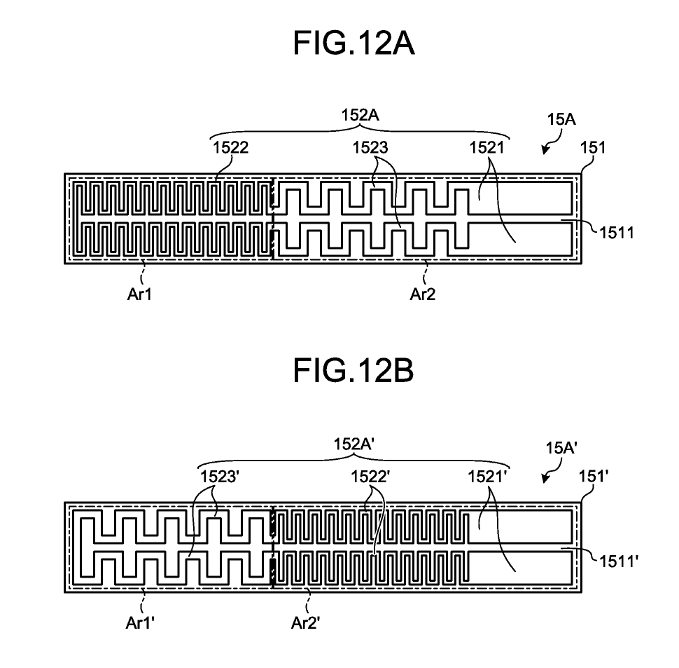

FIG. 12A is a diagram that illustrates a first heater 15A according to the second embodiment of the disclosure. Specifically, FIG. 12A is a diagram that corresponds to FIG. 9A.

As illustrated in FIG. 12A, the first heater 15A according to the second embodiment uses a first wiring pattern 152A having a different shape from the first wiring pattern 152 in the first heater 15 that is explained in the above-described first embodiment.

As illustrated in FIG. 12A, the first wiring pattern 152A includes a pair of first auxiliary heat-generating portions 1523 in addition to the pair of the first connecting portions 1521 and the first electric resistance pattern 1522 explained in the above-described first embodiment.

Furthermore, the length dimension (the length dimension in the longitudinal direction of the first board 151) of each of the first connecting portions 1521 in pair according to the second embodiment is shorter than that of each of the first connecting portions 1521 in pair explained in the above-described first embodiment, and it is set to be the same length dimension as that of each of the second connecting portions 1521′ in pair. Moreover, the first electric resistance pattern 1522 according to the second embodiment corresponds to the first heat-generating portion according to the disclosure as is the case with the above-described first embodiment.

As illustrated in FIG. 12A, the first auxiliary heat-generating portions 1523 in pair are provided at the second area Ar2 such that they are faced each other in a width direction of the first board 151. Furthermore, one end of one of the first auxiliary heat-generating portions 1523 is connected (electrically connected) to one of the first connecting portions 1521, serpentines from the end in a wavelike fashion with a constant line width, extends in the longitudinal direction of the first board 151, and the other end is connected (electrically connected) to one end of the first electric resistance pattern 1522. Moreover, one end of the other one of the first auxiliary heat-generating portions 1523 is connected (electrically connected) to the other one of the first connecting portions 1521, serpentines from the end in a wavelike fashion with a constant line width (the same line width as that of the one of the first auxiliary heat-generating portions 1523), extends in the longitudinal direction of the first board 151, and the other end is connected (electrically connected) to the other end of the first electric resistance pattern 1522.

According to the second embodiment, the line width of the first auxiliary heat-generating portions 1523 in pair is set smaller than the line width of the first connecting portions 1521 in pair and larger than the line width of the first electric resistance pattern 1522. Furthermore, the pitch of the first auxiliary heat-generating portions 1523 in pair (it is equivalent to the cycle of the waved first auxiliary heat-generating portion 1523) is set larger than the pitch of the first electric resistance pattern 1522. Moreover, the thickness dimension of each of the first connecting portions 1521 in pair, the first electric resistance pattern 1522, and the first auxiliary heat-generating portions 1523 in pair is set to be identical. That is, the resistance value of the first auxiliary heat-generating portions 1523 in pair per unit length in the longitudinal direction of the first board 151 is set larger than the resistance value of the first connecting portions 1521 in pair and smaller than the resistance value of the first electric resistance pattern 1522.

FIG. 12B is a diagram that illustrates a second heater 15A′ according to the second embodiment of the disclosure. Specifically, FIG. 12B is a diagram that corresponds to FIG. 9B.

As illustrated in FIG. 12B, the second heater 15A′ according to the second embodiment uses a second wiring pattern 152A′ having a different shape from that of the second wiring pattern 152′ in the second heater 15′ explained in the above-described first embodiment.

As illustrated in FIG. 12B, the second wiring pattern 152A′ includes a second auxiliary heat-generating portion 1523′ in addition to the pair of the second connecting portions 1521′ and the second electric resistance pattern 1522′ explained in the above-described first embodiment.

Furthermore, contrary to the second electric resistance pattern 1522′ explained in the above-described first embodiment, the second electric resistance pattern 1522′ according to the second embodiment is divided into two pieces with the center line in the width direction of the second board 151′ as a reference. Moreover, the pair of the second electric resistance patterns 1522′ corresponds to the first heat-generating portion according to the disclosure as is the case with the above-described first embodiment.

As illustrated in FIG. 12B, the second auxiliary heat-generating portion 1523′ is disposed at the first projection area Ar1′. Furthermore, one end of the second auxiliary heat-generating portion 1523′ is connected (electrically connected) to one of the second electric resistance patterns 1522′, serpentines from the end in a wavelike fashion with a constant line width, extends in a U shape that follows the outer edge shape of the second board 151′, and the other end is connected (electrically connected) to the other one of the second electric resistance patterns 1522′.

According to the second embodiment, the line width and the pitch of the second auxiliary heat-generating portion 1523′ are set to be the same as those of the first auxiliary heat-generating portion 1523. Furthermore, the thickness dimension of each of the second connecting portions 1521′ in pair, the second electric resistance patterns 1522′ in pair, and the second auxiliary heat-generating portion 1523′ is set to be identical. That is, the resistance value of the second auxiliary heat-generating portion 1523′ per unit length in the longitudinal direction of the second board 151′ is set larger than the resistance value of the second connecting portions 1521′ in pair and smaller than the resistance value of the second electric resistance patterns 1522′ in pair.

Moreover, in the “closed state” illustrated in FIG. 8, the first electric resistance pattern 1522 is faced the second auxiliary heat-generating portion 1523′. Also, the second electric resistance patterns 1522′ in pair are faced the first auxiliary heat-generating portions 1523 in pair, respectively.

The advantage similar to that of the above-described first embodiment is produced when the first and the second heaters 15A, 15A′ according to the above-described second embodiment are used.

Furthermore, the first auxiliary heat-generating portion 1523 is disposed at the second area Ar2 in the first heater 15A according to the second embodiment. Conversely, in the second heater 15A′ according to the second embodiment, the second auxiliary heat-generating portion 1523′ is disposed at the first projection area Ar1′.

For this reason, not only the first electric resistance pattern 1522 but also the second auxiliary heat-generating portion 1523′, which has a temperature lower than that of the first electric resistance pattern 1522, may apply thermal energy to the living tissue when it is held at the distal end side as described above. Similarly, not only the pair of the second electric resistance patterns 1522′ but also the first auxiliary heat-generating portion 1523, which has a temperature lower than that of the pair of the second electric resistance patterns 1522′, may apply thermal energy to the living tissue when it is held at the proximal end side as described above.

Thus, the first and the second heaters 15A, 15A′ according to the second embodiment may further reduce the treatment time for the living tissue.

### Third Embodiment

Next, a third embodiment of the disclosure is explained.

In the following explanation, the same configuration as that of the above-described first embodiment is attached with the same reference numeral, and its detailed explanations are omitted or simplified.

A treatment tool according to the third embodiment is different from the treatment tool 2 explained in the above-described first embodiment in the configurations of the first and the second heaters 15, 15′. Therefore, only the configurations of the first and second heaters according to the third embodiment are explained below.

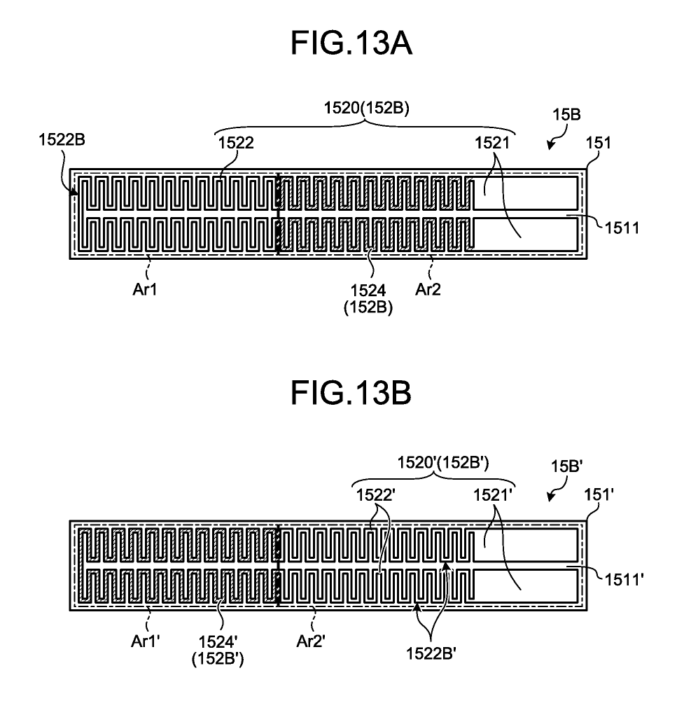

FIG. 13A is a diagram that illustrates a first heater 15B according to the third embodiment of the disclosure. Specifically, FIG. 13A is a diagram that corresponds to FIG. 9A.

As illustrated in FIG. 13A, the first heater 15B according to the third embodiment uses a first wiring pattern 152B different from the first wiring pattern 152 in the first heater 15 explained in the above-described first embodiment.

As illustrated in FIG. 13A, the first wiring pattern 152B includes a first wiring-pattern main body 1520 and a pair of first conductive portions 1524.

The first wiring-pattern main body 1520 is a portion that corresponds to the above-described first wiring pattern 152, and it includes the pair of the first connecting portions 1521 and the first electric resistance pattern 1522.

Furthermore, the length dimension (the length dimension in the longitudinal direction of the first board 151) of each of the first connecting portions 1521 in pair according to the third embodiment is shorter than that of each of the first connecting portions 1521 in pair explained in the above-described first embodiment, and the length dimension is set to be the same as that of each of the second connecting portions 1521′ in pair. Moreover, the length dimension (the length dimension in the longitudinal direction of the first board 151) of the first electric resistance pattern 1522 according to the third embodiment is longer than that of the first electric resistance pattern 1522 explained in the above-described first embodiment, and it is formed such that it extends across the first and the second areas Ar1, Ar2.

The pair of the first conductive portions 1524 is made of an electric conductive material, such as gold, silver, copper, or nickel (a material with a higher conductivity (a lower electric resistance value) than the first wiring-pattern main body 1520), and as illustrated in a diagonal line in FIG. 13A, and it is formed by plating or electrocasting on the first electric resistance pattern 1522 at the area that corresponds to the second area Ar2.

Specifically, the resistance value per unit length in the longitudinal direction of the first board 151 is smaller in this order: a portion 1522B of the first electric resistance pattern 1522 where the pair of the first conductive portions 1524 is not formed, the portion of the first electric resistance pattern 1522 where the pair of the first conductive portions 1524 are formed, and the pair of the first connecting portions 1521. Furthermore, the portion 1522B corresponds to the first heat-generating portion according to the disclosure (hereafter, the portion 1522B is described as the first heat-generating portion 1522B). Furthermore, the portion of the first electric resistance pattern 1522 where the pair of the first conductive portions 1524 is formed corresponds to the first auxiliary heat-generating portion according to the disclosure. That is, the pair of the first conductive portions 1524 is provided at the area of the first electric resistance pattern 1522 other than the first heat-generating portion 1522B.

FIG. 13B is a diagram that illustrates a second heater 15B′ according to the third embodiment of the disclosure. Specifically, FIG. 13B is a diagram that corresponds to FIG. 9B.

As illustrated in FIG. 13B, the second heater 15B′ according to the third embodiment uses a second wiring pattern 152B′ different from the second wiring pattern 152′ in the second heater 15′ explained in the above-described first embodiment.

As illustrated in FIG. 13B, the second wiring pattern 152B′ includes a second wiring-pattern main body 1520′ and a second conductive portion 1524′.

The second wiring-pattern main body 1520′ is of the same material and shape as those of the above-described first wiring-pattern main body 1520, and it includes the pair of the second connecting portions 1521′ and the second electric resistance pattern 1522′, each corresponding to the pair of the first connecting portions 1521 and the first electric resistance pattern 1522.

The second conductive portion 1524′ is made of an electric conductive material, such as gold, silver, copper, or nickel (a material with a higher conductivity (a lower electric resistance value) than the second wiring-pattern main body 1520′), and as illustrated in a diagonal line in FIG. 13B, it is formed by plating or electrocasting on the second electric resistance pattern 1522′ at the area that is positioned at the first projection area Ar1′.

Specifically, the resistance value per unit length in the longitudinal direction of the second board 151′ is smaller in this order: each portion 1522B′ of the second electric resistance pattern 1522′ where the second conductive portion 1524′ is not formed, the portion of the second electric resistance pattern 1522′ where the second conductive portion 1524′ is formed, and the pair of the second connecting portions 1521′. Furthermore, the portion 1522B′ corresponds to the second heat-generating portion according to the disclosure (hereafter, each of the portions 1522B′ is described as the pair of the second heat-generating portions 1522B′). Moreover, the portion of the second electric resistance pattern 1522′ where the second conductive portion 1524′ is formed corresponds to the second auxiliary heat-generating portion according to the disclosure. That is, the second conductive portion 1524′ is provided at the area of the second electric resistance pattern 1522′ other than the second heat-generating portion 1522B′.

Furthermore, in the “closed state” illustrated in FIG. 8, the first heat-generating portion 1522B is faced the second conductive portion 1524′. Moreover, the pair of the second heat-generating portions 1522B′ is faced the pair of the first conductive portions 1524.

The advantage similar to that of the above-described first and second embodiments is produced when the first and the second heaters 15B, 15B′ according to the above-described third embodiment are used.

### Fourth Embodiment

Next, a fourth embodiment of the disclosure is explained.

In the following explanation, the same structure as that in the above-described first embodiment is attached with the same reference numeral, and its detailed explanations are omitted or simplified.

A treatment tool according to the fourth embodiment is different from the treatment tool 2 explained in the above-described first embodiment in the configurations of the first and the second heaters 15, 15′. Therefore, only the configurations of the first and second heaters according to the fourth embodiment are explained below.

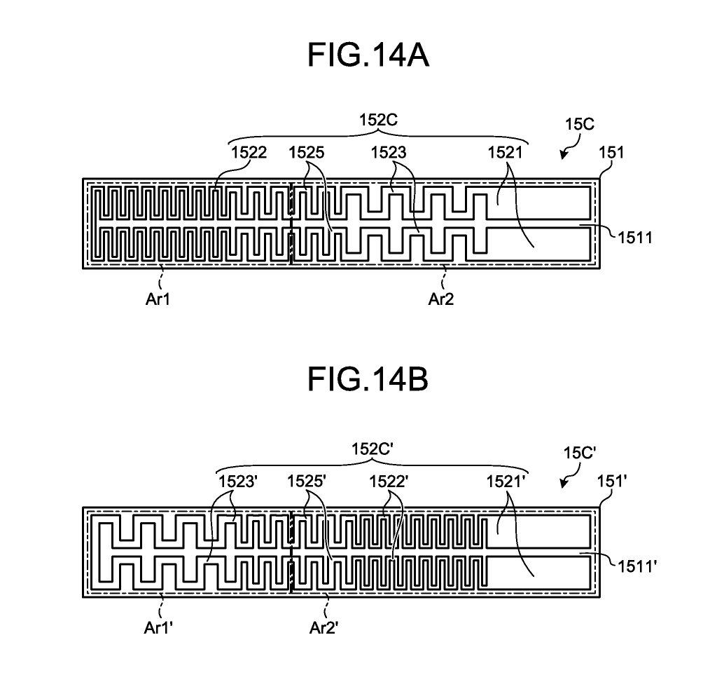

FIG. 14A is a diagram that illustrates a first heater 15C according to the fourth embodiment of the disclosure. Specifically, FIG. 14A is a diagram that corresponds to FIG. 9A.

As illustrated in FIG. 14A, the first heater 15C according to the fourth embodiment uses a first wiring pattern 152C having a different shape from the first wiring pattern 152 in the first heater 15 explained in the above-described first embodiment.

As illustrated in FIG. 14A, the first wiring pattern 152C includes a pair of first intermediate heat-generating portions 1525 as well as the pair of the first connecting portions 1521, the first electric resistance pattern 1522, and the pair of the first auxiliary heat-generating portions 1523 explained in the above-described second embodiment.

Here, contrary to the first electric resistance pattern 1522 explained in the above-described second embodiment, the first electric resistance pattern 1522 according to the fourth embodiment is formed such that each end at the proximal end side (the right side in FIG. 14A) is located away from the second area Ar2 by a predetermined distance. Furthermore, contrary to the pair of the first auxiliary heat-generating portion 1523 explained in the above-described second embodiment, the pair of the first auxiliary heat-generating portions 1523 according to the fourth embodiment is formed such that each end at the distal end side (the left side in FIG. 14A) is located away from the first area Ar1 by a predetermined distance.

Moreover, the first electric resistance pattern 1522 according to the fourth embodiment corresponds to the first heat-generating portion according to the disclosure as is the case with the above-described second embodiment.

As illustrated in FIG. 14A, the first intermediate heat-generating portions 1525 in pair are provided such that they are faced each other in the width direction of the first board 151. Furthermore, one end of one of the first intermediate heat-generating portions 1525 is connected (electrically connected) to one of the first auxiliary heat-generating portions 1523, serpentines from the end in a wavelike fashion with a constant line width, extends in the longitudinal direction of the first board 151, and the other end is connected (electrically connected) to one end of the first electric resistance pattern 1522. Moreover, one end of the other one of the first intermediate heat-generating portions 1525 is connected (electrically connected) to the other one of the first auxiliary heat-generating portions 1523, serpentines from the end in a wavelike fashion with a constant line width (the same line width as that of the one of the first intermediate heat-generating portions 1525), extends in the longitudinal direction of the first board 151, and the other end is connected (electrically connected) to the other end of the first electric resistance pattern 1522. Specifically, the pair of the first intermediate heat-generating portions 1525 is positioned between the first electric resistance pattern 1522 and the pair of the first auxiliary heat-generating portions 1523, and it is provided so as to extend across the first and the second areas Ar1, Ar2.

According to the fourth embodiment, the line width of the first intermediate heat-generating portions 1525 in pair is set smaller than the line width of the first auxiliary heat-generating portions 1523 in pair and larger than the line width of the first electric resistance pattern 1522. Furthermore, the pitch of the first intermediate heat-generating portions 1525 in pair (it is equivalent to the cycle of the waved first intermediate heat-generating portion 1525) is set larger than the pitch of the first electric resistance pattern 1522 and smaller than the pitch of the first auxiliary heat-generating portion 1523. Furthermore, the thickness dimension of each of the first electric resistance pattern 1522, the first auxiliary heat-generating portions 1523 in pair, and the first intermediate heat-generating portions 1525 in pair is set to be identical. That is, the resistance value of the pair of the first intermediate heat-generating portions 1525 per unit length in the longitudinal direction of the first board 151 is set larger than that of the pair of the first auxiliary heat-generating portions 1523 and smaller than the resistance value of the first electric resistance pattern 1522.

FIG. 14B is a diagram that illustrates a second heater 15C′ according to the fourth embodiment of the disclosure. Specifically, FIG. 14B is a diagram that corresponds to FIG. 9B.

As illustrated in FIG. 14B, the second heater 15C′ according to the fourth embodiment uses a second wiring pattern 152C′ having a different shape from the second wiring pattern 152′ in the second heater 15′ explained in the above-described first embodiment.

As illustrated in FIG. 14B, the second wiring pattern 152C′ includes a pair of second intermediate heat-generating portions 1525′ as well as the pair of the second connecting portions 1521′, the pair of the second electric resistance pattern 1522′, and the second auxiliary heat-generating portion 1523′ explained in the above-described second embodiment.

Furthermore, contrary to the second auxiliary heat-generating portion 1523′ explained in the above-described second embodiment, the second auxiliary heat-generating portion 1523′ according to the fourth embodiment is formed such that each end at the proximal end side (the right side in FIG. 14B) is located away from the second projection area Ar2′ by a predetermined distance. Moreover, contrary to the pair of the second electric resistance patterns 1522′ explained in the above-described second embodiment, the pair of the second electric resistance patterns 1522′ according to the fourth embodiment is formed such that each end at the distal end side (the left side in FIG. 14B) is located away from the first projection area Ar1′ by a predetermined distance.

Also, the pair of the second electric resistance patterns 1522′ according to the fourth embodiment corresponds to the second heat-generating portion according to the disclosure as is the case with the above-described second embodiment.

As illustrated in FIG. 14B, the second intermediate heat-generating portions 1525′ in pair are provided such that they are opposed to each other in the width direction of the second board 151′. Furthermore, one end of one of the second intermediate heat-generating portions 1525′ is connected (electrically connected) to one of the second electric resistance patterns 1522′, serpentines from the end in a wavelike fashion with a constant line width, extends in the longitudinal direction of the second board 151′, and the other end is connected (electrically connected) to one end of the second auxiliary heat-generating portion 1523′. Moreover, one end of the other one of the second intermediate heat-generating portion 1525′ is connected (electrically connected) to the other one of the second electric resistance patterns 1522′, serpentines from the end in a wavelike fashion with a constant line width (the same line width as that of the one of the second intermediate heat-generating portion 1525′), extends in the longitudinal direction of the second board 151′, and the other end is connected (electrically connected) to the other end of the second auxiliary heat-generating portions 1523′. That is, the pair of the second intermediate heat-generating portions 1525′ is positioned between the second auxiliary heat-generating portion 1523′ and the pair of the second electric resistance patterns 1522′, and it is provided so as to extend across the first and the second projection areas Ar1′, Ar2′.

According to the fourth embodiment, the line width and the pitch of the pair of the second intermediate heat-generating portions 1525′ is set to be the same as those of the first intermediate heat-generating portion 1525. Furthermore, the thickness dimension of each of the second electric resistance patterns 1522′ in pair, the second auxiliary heat-generating portion 1523′, and the second intermediate heat-generating portions 1525′ in pair is set to be identical. That is, the resistance value of the pair of the second intermediate heat-generating portions 1525′ per unit length in the longitudinal direction of the second board 151′ is set larger than the resistance value of the second auxiliary heat-generating portion 1523′ and smaller than the resistance value of the pair of the second electric resistance patterns 1522′.

Furthermore, in the “closed state” illustrated in FIG. 8, the first electric resistance pattern 1522 is faced the second auxiliary heat-generating portion 1523′. Moreover, the pair of the second electric resistance patterns 1522′ is faced the pair of the first auxiliary heat-generating portions 1523. Further, the pair of the first intermediate heat-generating portions 1525 is faced the pair of the second intermediate heat-generating portions 1525′.

The advantage similar to that of the above-described first and second embodiments is produced when the first and the second heaters 15C, 15C′ according to the above-described fourth embodiment are used.

### Other Embodiments

Although the embodiments for implementing the disclosure have been explained above, the disclosure should not be limited to only the above-described first to fourth embodiments.

With regard to the opening/closing system for opening and closing the first and the second jaws 11, 11′ according to the above-described first to fourth embodiments, not only the opening/closing system explained in the above-described first to fourth embodiments but also other systems may be used. Specifically, not only the structure for moving (opening/closing) both the first and the second jaws 11, 11′ as in the above-described first to fourth embodiments but also the structure for moving (opening/closing) one of them while the other one is fixed may be used.

According to the above-described first to fourth embodiments, thermal energy is used as energy applied to the living tissue; however, this is not a limitation, and it is possible to use the structure for further applying high-frequency energy or ultrasound energy other than thermal energy to the living tissue.

According to the above-described first, second and fourth embodiments, the resistance value per unit length in the longitudinal direction of the first board 151 is changed by changing the line width or the pitch for the portion 1522, which corresponds to the first heat-generating portion according to the disclosure, the first auxiliary heat-generating portion 1523, the first intermediate heat-generating portion 1525, and the pair of the first connecting portions 1521; however, this is not a limitation. The resistance value per unit length in the longitudinal direction of the first board 151 may be changed by, for example, changing any one of the line width and the pitch, changing the thickness dimension, or changing the material. Furthermore, the same holds for the portion 1522′, which corresponds to the second heat-generating portion according to the disclosure, the second auxiliary heat-generating portion 1523′, the second intermediate heat-generating portion 1525′, and the pair of the second connecting portions 1521′.

According to the above-described first to fourth embodiments, the first board 151 may be omitted and the first wiring pattern 152 (152A, 152B) may be directly mounted on the first holding surface 1111. In this case, the first jaw 11 is formed of an insulating material in the same manner as the first board 151 or is formed of an electric conductive material and is provided with insulating coating to be electrically insulated from the first wiring pattern 152 (152A, 152B). Furthermore, the same holds for the second wiring pattern 152′ (152A′, 152B′).

According to the above-described first to fourth embodiments, the applied-current controller 35 calculates the first heater temperature based on the resistance value of the first wiring pattern 152, 152A to 152C in whole when applying current and executes applied-current control on the first wiring pattern 152, 152A to 152C so that the first heater temperature becomes the target temperature; however, this is not a limitation. For example, the first heater temperature may be calculated based on only the resistance value of the portion 1522, 1522B corresponding to the first heat-generating portion according to the disclosure when applying current and applied-current control may be executed on the first wiring pattern 152, 152A to 152C so that the first heater temperature becomes the target temperature. Also, with regard to the second heater temperature, the second heater temperature may be calculated based on only the resistance value of the portion 1522′, 1522B′ corresponding to the second heat-generating portion according to the disclosure when applying current and applied-current control may be executed on the second wiring pattern 152′, 152A′ to 152C′ so that the second heater temperature becomes the target temperature.

Furthermore, according to the above-described first to fourth embodiments, when the distal end part (the holding portion 7 and part of the shaft 6) of the treatment tool 2 is configured as a disposable portion that is disposed of after use, the distal end part corresponds to the treatment tool according to the disclosure.

With the treatment tool and the treatment system according to the disclosure, there are advantages such that the living tissue may be heated with the desired temperature and the treatment time may be reduced.

### BRIEF DESCRIPTION OF DRAWINGS

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a first jaw including a first holding surface;
a second jaw including a second holding surface to hold living tissue with the first holding surface;
a first wiring pattern that is provided on the first holding surface and that includes a first heat-generating portion where a resistance value per unit length in a longitudinal direction connecting a distal end and a proximal end of the first jaw is higher than resistance values of other areas, the first heat-generating portion being configured to generate heat with an applied current;
a first heating plate that is disposed to face the first holding surface, the first heating plate being configured to transmit heat from the first wiring pattern to the living tissue by bringing into contact with the living tissue;
a second wiring pattern that is provided on the second holding surface and that includes a second heat-generating portion where a resistance value per unit length in a longitudinal direction connecting a distal end and a proximal end of the second jaw is higher than resistance values of other areas, the second heat-generating portion being configured to generate heat with an applied current; and
a second heating plate that is disposed to face the second holding surface, the second heating plate being configured to transmit heat from the second wiring pattern to the living tissue by bringing into contact with the living tissue, wherein
the first heat-generating portion is provided at a first area when the first holding surface is divided into two areas where the first area and a second area are arranged parallel in the longitudinal direction connecting the distal end and the proximal end of the first jaw, and
the second heat-generating portion is provided at a second projection area when the second holding surface is divided into two areas where a first projection area onto which the first area is projected and the second projection area onto which the second area is projected in a closed state where the first holding surface and the second holding surface are faced each other.

2. The treatment tool according to claim 1, wherein
the first wiring pattern further includes a first auxiliary heat-generating portion that is disposed at the second area and where a resistance value per unit length in the longitudinal direction connecting the distal end and the proximal end of the first jaw is lower than a resistance value of the first heat-generating portion, the first auxiliary heat-generating portion being configured to generate heat with an applied current and
the second wiring pattern further includes a second auxiliary heat-generating portion that is disposed at the first projection area and where a resistance value per unit length in the longitudinal direction connecting the distal end and the proximal end of the second jaw is lower than the resistance value of the second heat-generating portion, the second auxiliary heat-generating portion being configured to generate heat with an applied current.

3. The treatment tool according to claim 2, wherein
the first wiring pattern further includes a first intermediate heat-generating portion that is disposed between the first heat-generating portion and the first auxiliary heat-generating portion so as to extend across the first area and the second area and where a resistance value per unit length in the longitudinal direction connecting the distal end and the proximal end of the first jaw is lower than the resistance value of the first heat-generating portion and higher than the resistance value of the first auxiliary heat-generating portion, the first intermediate heat-generating portion being configured to generate heat with an applied current and
the second wiring pattern further includes a second intermediate heat-generating portion that is disposed between the second heat-generating portion and the second auxiliary heat-generating portion so as to extend across the first projection area and the second projection area and where a resistance value per unit length in the longitudinal direction connecting the distal end and the proximal end of the second jaw is lower than the resistance value of the second heat-generating portion and higher than the resistance value of the second auxiliary heat-generating portion, the second intermediate heat-generating portion being configured to generate heat with an applied current.

4. The treatment tool according to claim 1, wherein
the first wiring pattern includes:
a first wiring-pattern main body including the first heat-generating portion; and
a first conductive portion that is composed of a conductive material and that is provided at an area on the first wiring-pattern main body other than the first heat-generating portion, and
the second wiring pattern includes:
a second wiring-pattern main body including the second heat-generating portion; and
a second conductive portion that is composed of a conductive material and is provided at an area on the second wiring-pattern main body other than the second heat-generating portion.

5. The treatment tool according to claim 2, wherein
the first wiring pattern includes:
a first wiring-pattern main body including the first heat-generating portion; and
a first conductive portion that is composed of a conductive material and that is provided at an area on the first wiring-pattern main body other than the first heat-generating portion, and
the second wiring pattern includes:
a second wiring-pattern main body including the second heat-generating portion; and
a second conductive portion that is composed of a conductive material and is provided at an area on the second wiring-pattern main body other than the second heat-generating portion.

6. The treatment tool according to claim 3, wherein
the first wiring pattern includes:
a first wiring-pattern main body including the first heat-generating portion; and
a first conductive portion that is composed of a conductive material and that is provided at an area on the first wiring-pattern main body other than the first heat-generating portion, and
the second wiring pattern includes:
a second wiring-pattern main body including the second heat-generating portion; and
a second conductive portion that is composed of a conductive material and is provided at an area on the second wiring-pattern main body other than the second heat-generating portion.

7. A treatment system comprising:
the treatment tool according to claim 1; and
an applied-current controller configured to apply a current to each of the first wiring pattern and the second wiring pattern, calculate a temperature based on a resistance value of each of the first wiring pattern and the second wiring pattern when applying the current, and execute an applied-current control such that the temperature becomes a target temperature.

8. A treatment system comprising:
the treatment tool according to claim 2; and
an applied-current controller configured to apply a current to each of the first wiring pattern and the second wiring pattern, calculate a temperature based on a resistance value of each of the first wiring pattern and the second wiring pattern when applying the current, and execute an applied-current control such that the temperature becomes a target temperature.

9. A treatment system comprising:
the treatment tool according to claim 3; and
an applied-current controller configured to apply a current to each of the first wiring pattern and the second wiring pattern, calculate a temperature based on a resistance value of each of the first wiring pattern and the second wiring pattern when applying the current, and execute an applied-current control such that the temperature becomes a target temperature.

10. A treatment system comprising:
the treatment tool according to claim 4; and
an applied-current controller configured to apply a current to each of the first wiring pattern and the second wiring pattern, calculate a temperature based on a resistance value of each of the first wiring pattern and the second wiring pattern when applying the current, and execute an applied-current control such that the temperature becomes a target temperature.

11. The treatment system according to claim 7, wherein the applied-current controller is configured to calculate the temperature based on a resistance value of each of the first heat-generating portion and the second heat-generating portion while a current is applied to each of the first wiring pattern and the second wiring pattern, and execute an applied-current control such that the temperature becomes a target temperature.

12. The treatment system according to claim 8, wherein the applied-current controller is configured to calculate the temperature based on a resistance value of each of the first heat-generating portion and the second heat-generating portion while a current is applied to each of the first wiring pattern and the second wiring pattern, and execute an applied-current control such that the temperature becomes a target temperature.

13. The treatment system according to claim 9, wherein the applied-current controller is configured to calculate the temperature based on a resistance value of each of the first heat-generating portion and the second heat-generating portion while a current is applied to each of the first wiring pattern and the second wiring pattern, and execute an applied-current control such that the temperature becomes a target temperature.

14. The treatment system according to claim 10, wherein the applied-current controller is configured to calculate the temperature based on a resistance value of each of the first heat-generating portion and the second heat-generating portion while a current is applied to each of the first wiring pattern and the second wiring pattern, and execute an applied-current control such that the temperature becomes a target temperature.
